(19) United States
(12) Patent Application Publication
(10) Pub. No.: US 2010/0022988 A1
(43) Pub. Date: Jan. 28, 2010

(54) INFUSION SET WITH A DATA STORAGE DEVICE

(76) Inventors: Micaela Wochner, Forch (CH); Jurg Liniger, Ostermundigen (CH); Martin Wyss, Burgdorf (CH)

Correspondence Address: Dorsey & Whitney LLP, IP Department, ATTN: Disetronic Licensing AG, 50 South Sixth Street, Suite 1500, Minneapolis, MN, 55402-1498, US

(21) Appl. No.: 12/489,839

(22) Filed: Jun. 23, 2009

Related U.S. Application Data
(63) Continuation of application No. 12/266,871, filed on Nov. 7, 2008, now abandoned.
Application No. 12/266,871 is a continuation of application No. PCT/CH2007/000204, filed on Apr. 27, 2007.

(30) Foreign Application Priority Data
May 10, 2006 (CH) 751/06

(51) Int. Cl. A61M 5/14; A61M 25/16; H04Q 5/22

(52) U.S. Cl. 604/506; 604/93.01; 604/533; 340/10.1

(57) ABSTRACT

An infusion device, an infusion set and a system and method involving one or the other and/or both, wherein the infusion device interacts with the infusion set, and/or wherein the device, set, system and method ensure that deviations from the therapeutically suitable operating state of an infusion set attached to the infusion device can be detected and rectified in an automated manner or with the assistance of the person using the infusion device.

### CROSS REFERENCE TO RELATED APPLICATIONS

This application is a continuation of PCT application No: PCT/CH2007/000204 filed Apr. 27, 2007, which claims priority to Swiss Patent Application No: 751/06 filed May 10, 2006, both of which are incorporated herein by reference.

### TECHNICAL FIELD

The present invention relates devices for delivering, injecting, infusing, administering or dispensing a substance, and to methods of making and using such devices. More particularly, it relates to an infusion set for a device that is worn on the outside of the body and continuously administers a medical or pharmaceutical active substance into organic tissue.

### BACKGROUND

Devices of this kind, for example consisting of an infusion device (e.g., and infusion pump) and of an infusion set, are used in ambulant insulin pump treatment for stabilizing the blood sugar levels in diabetic patients. A conventional infusion set is composed of a connector which is provided on or connected to the infusion device and which is connected fluidically to a catheter head via a catheter tube. A cannula is arranged on the catheter head and is inserted by the patient into the subcutaneous body tissue. The continuous administration of active substance by means of an infusion device and infusion set is subject to a number of basic requirements in order to ensure the success of the treatment. For hygiene reasons, in order to avoid insulin resistance and to prevent allergic reactions, an infusion set has to be replaced with varying localization after approximately 2-3 days of use and can be used just once. Suitable authorized infusion sets are offered by the manufacturers of infusion appliances or by approved manufacturers and are presented in sterile packages.

The replacement of the infusion set is carried out by the person wearing or using the infusion device, e.g., insulin pump. It is therefore the responsibility of the patient to ensure that an authorized infusion set that has not been previously used and that has not passed its expiry date is applied. However, a large number of infusion sets are commercially available that have standard Luer lock connectors and thus in principle permit the connection between infusion device and infusion set, even though they are not suitable for use with a certain infusion devices or are not authorized by the manufacturer.

The continuous administration of active substance by means of an infusion appliance and infusion set is subject to a number of additional requirements in order to ensure the success of the treatment and to ensure that an infusion set conforms with the therapy.

For an infusion set appropriate for a particular therapy, it is required that the fluid line is completely filled with active substance, and free from bubbles, before the infusion set is applied and before the cannula is inserted into the body tissue. This procedure is generally referred to as the priming procedure. Because of the variety of models of infusion sets with different tube lengths and filling volumes, the priming procedure has to be performed manually by the patient and monitored. A priming procedure is considered to have been successfully concluded when a droplet of the medicament to be administered appears at the tip of the as yet uninserted cannula. Monitoring the emergence of the droplet by sight places high demands on the visual acuity of the diabetes patient, and is often asking too much, given the symptoms typical of diabetes. If too much liquid is introduced as a result of so-called overpriming, there is the danger of uncontrolled contamination of the environment with active substance.

Because of their routine nature, such tasks that need to be mastered by the person wearing or using the pump may increasingly lead to safety-critical situations.

Infusion sets with integrated data memory and identification systems to provide protection against use of unauthorized or already used infusion sets are known. For example, an accessory part attachable to an administration appliance in medical device having may have coded or uncoded information regarding previous use, and multiple uses of the accessory part may be prevented.

A fluid delivery system as part of an infusion set with a data memory is also known, and includes the individual flow resistance and circulation values of the particular infusion set to permit a more precise administration of the fluidic active substance.

A RFID data memory containing the administration data prescribed by the physician is also known, which ensures that the correct medicament is administered in the correct dose.

However, an infusion set with a data memory that is capable of communicating to and/or with an infusion device to execute more than one function is not known.

### SUMMARY

One of the objects of the present invention concerns the exchange and operation of an infusion device interacting with an infusion set to ensure that deviations from the therapeutically suitable operating state of an infusion set attached to an infusion device can be detected and rectified in an automated manner, or with the assistance of the person wearing or using the pump.

### BRIEF DESCRIPTION OF THE DRAWINGS

FIG. 1 is a perspective view of an infusion set coupled to an infusion device such as an insulin pump, where the data memory is situated near the fluidic connector.

FIG. 2 is a perspective view of an infusion set coupled to an infusion device such as an insulin pump, where the data memory is situated on the catheter head.

FIG. 3 is a perspective view of an infusion set of FIG. 2 where the data memory is situated in the catheter head, wherein the catheter tubing is removed from the catheter head.

Skilled artisans appreciate that elements in the figures are illustrated for simplicity and clarity and have not necessarily been drawn to scale. For example, the dimensions of some of the elements in the figure may be exaggerated relative to other elements to help improve understanding of the embodiment(s) of the present invention. Also, it should be appreciated that the drawings illustrate only some embodiments of the present invention.

### DETAILED DESCRIPTION

For the purposes of promoting an understanding of the principles of the present invention, reference will now be made to a number of illustrative embodiments as shown in the attached drawings.

In one embodiment, the present invention comprises an infusion device or an administration device (terms used interchangeably through out the text), such as an insulin pump, and an infusion set, wherein the infusion set is removably connected to the infusion device. The infusion set, according to the present invention, is based, in terms of its fluidic configuration, on embodiments that are known in the art. The connection between the infusion set and an infusion device is provided by a Luer lock connector, which closes and opens in the form of a threaded closure piece. Proprietary connectors with the same function are also known in the art.

An infusion device may include an insulin pump for continuous delivery of medical active substances and a coupled infusion set. A quantity of insulin prescribed by the physician is discharged by the insulin pump to therapeutically stabilize the blood sugar level. The insulin pump is compact and can be worn discreetly near the body. The spatial distance between the optimum wearing position of the insulin pump and the injection site is overcome by the flexible catheter line of the infusion set. An infusion device cooperating with the infusion set according to the present invention comprises a processor-controlled metering device with which the active substance dose is dispensed. The nature of the dosed administration is known in the art, and insulin pumps that can communicate with external devices via data or command interfaces are known.

In accordance with one embodiment of the present invention, the infusion set is provided with a data memory. The data memory on or associated with the infusion set provides data to the infusion device to which the infusion set is connected. If, on the basis of the data present on the data memory of the infusion set, it is initially found that an infusion set is present which is authorized with respect to the infusion device, suitable instructions are delivered to the infusion device. If no authorized infusion device is present, use with the infusion device is prevented or suitably restricted.

A further advantage of the interaction created between the infusion set and the infusion device lies in the possibility of transmitting information concerning safety-critical operating states, generally referred to hereinbelow as “not conforming with the therapy.” Such operating states, which may compromise the successful outcome of the therapy, are:

a leak in the fluid channel of the infusion set;

an occlusion in the fluid channel of the infusion set, for example if the catheter tube is squeezed together by body parts bearing on it;

the connection between the infusion set and the infusion device has not been made correctly;

the connection between head and tube of the catheter head has not been made correctly;

the infusion set does not sit correctly on the body.

The data memory on the infusion set which contains suitable data is transmitted/communicated to the infusion device via a data exchange device that is provided on the infusion device. Suitable data on the data memory can be information such as “Infusion set type,” from which a priming volume for the catheter tube can be derived, “Catheter tube length” or “Filling volume for the priming procedure.”

The data memories that can be used may be overwritable and non-overwritable information carriers of the kind which, in their main configurations, are generally known as RAM or ROM memories. ROM logic memory may be hard-wired, or memory may be manually configurable DIP switches, such as RAM memory. In the configuration according to the invention, ROM data memories contain information predefined by the manufacturer, such as serial number, expiry date, tube length, filling volume of the infusion set. One particular configuration is the WORM data memory (write-once-read-many times) which allows the data carrier to be written once with information. After one write operation, it can then only be read.

RAM memories, in a configuration according to the present invention, are to be used to store information relating to alarm states and operating states, such as, for example, “Occlusion detected,” “Leak detected,” “Infusion set not correctly connected to pump” or “Infusion set not correctly connected between catheter head and catheter tube,” and are provided with the updated values.

In one embodiment, sensors with specific tasks and/or configurations are located on the infusion set and are designed to detect specific operating states and, as a result, to supply an electrical output signal. Such detectors may serve as occlusion detectors, connection detectors or leak detectors. In contrast to a sensor, a detector provides the specific status information in binary form. Thus, for example, an occlusion detector presents its output information in true/false form. The status “True” corresponds in this case to the statement “Occlusion detected” or vice versa.

To deliver the information from the data memory to the infusion device, for example to an insulin pump, a data exchange device is provided. Suitable date exchange devices and methods include, for example, wireless transmission technology with transmission protocols that permit coded or uncoded data transmission modes.

In some embodiments of the present invention, data storage and data exchange are effected by RFID (radio frequency identification) technology, also used in near-range identification systems.

In other embodiments, data storage and data exchange are based on optical transfer systems or identification systems. Unidirectional optical methods of identifying data include barcode reading systems. IrDA (infrared data association), optical transmission methods, or developments such as 2D barcodes or hologram systems may also be used.

Some infusion sets allow the user to temporarily disconnect the catheter tube from the catheter head so the user or patient can remove the pump from his or her body, for example when bathing or showering. In such embodiments, the data memory is for this purpose divided into two autonomous data memory/transponder units. One data memory/transponder unit is situated on the proximal section and one on the distal section of an infusion set. In this way, it is also possible to determine whether the connectable units are still present in the initial configuration.

In another embodiment of the present invention, the data transfer is based on an optical identification method. A reader, for example a barcode scanner integrated in the infusion device, allows the information to be read out from the infusion set, when so required. The barcode in this case assumes the role of an optical ROM memory.

If an optical transfer method is used, the data memory may be situated on the distal section of the catheter head, on the proprietary or Luer lock connector, on a sealing wrapper applied to the infusion set, or on the infusion set pack.

In another embodiment of the present invention, data transfer may be wire-based. Electrical leads and corresponding contact connections on the connector may be responsible for transferring data signals needed for the data exchange. Such wire-based data transfer may only require low signal currents, which means that the energy consumption of the supplying administration device is relatively small. The data memories present on the infusion set as hard-wired memory units or DIP switches are in this case connected electrically to the control unit.

In mechanical embodiments of the present invention, mechanical coding cams in the connector likewise form a memory configuration.

Accordingly, the interactivity between infusion device and infusion set permits the use of numerous methods that are able to enhance the safety of the therapy and patient safety and to increase user friendliness. Such interactivity involves the identification of the presence of an authorized or unauthorized infusion set. Successful identification of an authorized infusion set enables the capabilities of the infusion device, which may be altered, initiated or expanded, or specific sequences can be instigated. The results of all the identification procedures can be logged on the infusion device and are available for analyses of traceability.

The present invention comprises method embodiments. One such method embodiment for the expansion or initiation of capabilities of the infusion device is the “autopriming” that is permitted for the first time by the present invention. In this method, data memory information of the infusion set that define a priming volume or that make it possible to determine a priming volume are read out, when required, i.e. after initial connection of a new infusion set. If the result obtained is recognized as valid, an automated priming procedure can be triggered on the infusion device, for example, after a command acknowledgement by the patient.

The critical volume of liquid for a priming procedure, defined by the catheter length and by the throughflow surface area of the catheter tube, can be retrieved from the data memory either directly as volume or via a constant, in the sense of data processing.

In this method, the priming volume can be determined from the type designation information of the infusion set. The type-specific priming volumes can be referenced on the insulin pump or stored in a so-called look-up table on the administration device.

In another method, with very stringent demands in respect of the precision of the priming volume, the required priming volume is determined by the manufacturer for each individual type and is stored as a parameter on the infusion set.

In another embodiment, a priming detector, located on the infusion set in immediate proximity to the cannula outlet, monitors the filling of the infusion set. When a priming procedure is initiated by the person wearing or using the pump, the priming sensor detects under real-time conditions the arrival of the medicament fluid at the sensor position. Taking into account the residual volume between sensor position and cannula outlet, the priming procedure is terminated. The output signal of the priming sensor is delivered as feedback to the control unit via the data memory and the data exchange device. A conductivity sensor in the fluidic cannula section with a defined threshold value characteristic represents one simple possibility of a so-called priming detector.

In another method embodiment according to the present invention, when the wearing or use period of the infusion set has expired, the data memory is influenced by the infusion device in such a way that further use is made impossible. In one embodiment, the corresponding information is stored in a WORM data memory which, after one write operation, no longer permits a status change. In this way, the infusion set is as it were invalidated in software.

In another method embodiment, the identification of an incorrect connection between administration device and infusion set is provided. If the connection is correct, an electrical or mechanical connection is established or interrupted that represents suitable status information. In an infusion set is identified that has not been correctly connected, an appropriate message appears on the infusion device.

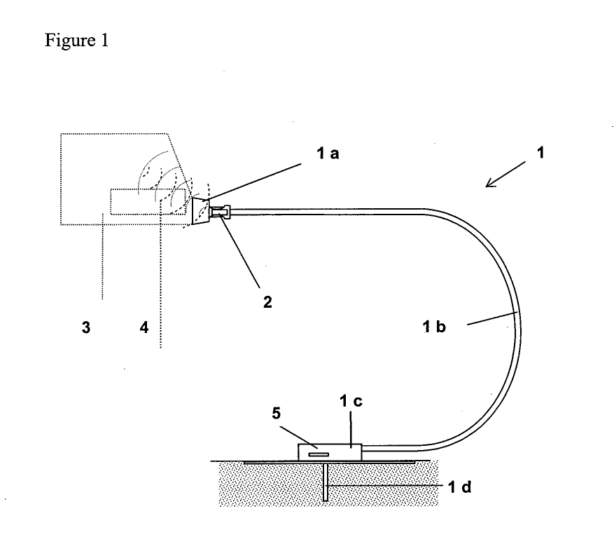

With reference to the drawings, FIG. 1 shows an infusion set (1) including connector (1a), tubing (1b), catheter head (1c) and cannula (1d) for administering medication into the body of an animal when cannula (1d) is inserted into the body tissue. The infusion set (1) includes data memory (2) for exchanging data with the infusion device (3) and is situated near the fluidic connector (1a). Data from data memory (2) may be transmitted to the infusion device (3) via a read and control device (4), e.g., a data exchange device, where deviations of the configuration of the infusion set (1) to a pre-defined therapy may be identified. In instances where a deviation in configuration of the infusion set (1) is detected, read and control device (4) may render the infusion set (1) non-operational, e.g., the infusion set (1) and infusion device (3) are not operable due to the infusion device (3) blocking medication from being dispensed into the infusion set (1). In instances where a deviation in configuration of the infusion set (1) is not detected, read and control device (4) may automatically establish an operational state of the infusion set (1), e.g., the combination of the infusion set (1) and the infusion device (3) are operable to dispense and deliver medication. Alternatively, in the absence of a configuration error, a user may manually establish an operational state of the infusion set (1). Thus, read and control device (4) of infusion device (3) may be associated with at least three medication delivery states, an initial state before data is received from data memory (2), a blocking state when deviation in configuration data is received from memory (2), and a dispensing state when the data received from memory (2) indicates no deviation in configuration is present.

In FIG. 1, for the purpose of exchanging data between data memory (2) and read and control device (4), data memory (2) is connected with an RFID close-coupling system to the read and control device (4). In close-coupling systems, the range is approximately 1 cm, and as a result of the short distance between the data memory (2) and the read and control device (4), the data transfer can be effected via an inductive or capacitive coupling between reader and transponder. By virtue of low signal levels and the possibility of good screening of the signal path, a high standard of safety can be guaranteed. An advantage of the rigid arrangement between reader and transponder is that there is a constant signal level. Because of the short transmission distance with low signal levels, the energy requirement for the transponder/data memory device on the infusion set (1) is low and can be taken from the coupling field or transfer field.

On the catheter head (1c) there are one or more sensors (5) with specific tasks, for example an occlusion detector, a leakage detector, or a priming detector which is in data-processing communication with the data memory (2) and transfers its current status information to the data memory (2). The data transfer to the data memory (2) takes place via an electrical connection that is integrated in the catheter tube. The voltage supply, effected either via electrical contacts on the connector or by removal from the coupling field, is fed to the sensor on the catheter head via a cable connection routed through the catheter tube.

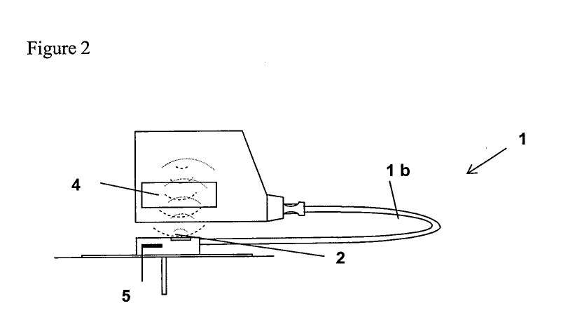

FIG. 2 shows another embodiment of an infusion device for a metered administration of a medical substance (3) with a connected infusion set (1), according to a further embodiment, using a close-coupling RFID system with a memory/transponder unit (2) which is situated on the catheter head. When the desired identification procedure between infusion set (1) and infusion device is to be permitted, the reader device on the infusion device (4) must be guided to the catheter head with the integrated RFID tag and positioned. If an automatic priming procedure is to be performed, an identification must first be carried out to ascertain whether an infusion set (1) has been attached that has suitable data that describe a priming volume or make it possible to derive a priming volume. If a validity check establishes that the information data permit safe priming, the catheter tube is filled with the active substance, with the cannula not yet inserted into the body tissue. A priming sensor arranged on the catheter head transmits, via the RAM data memory, the status signal that indicates a successful priming procedure.

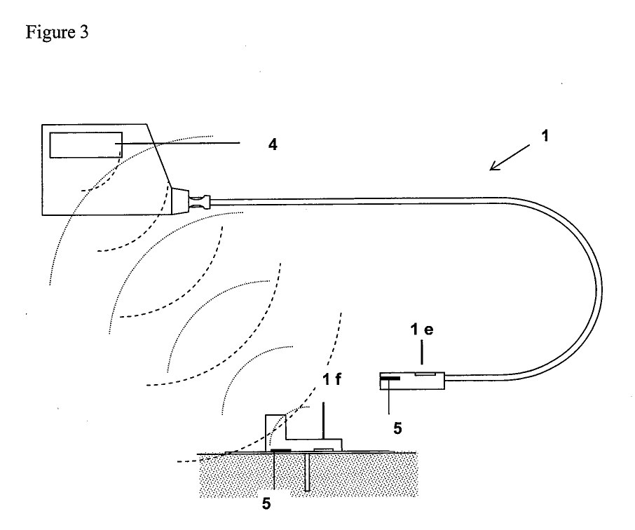

FIG. 3 shows an infusion set (1) further including a catheter head (1f), which allows the person wearing or using the pump to briefly disconnect the catheter tube from the catheter head, for example in order to be able to remove the pump and catheter tube before taking a bath or shower. The fluidic separation is effected on the catheter head, the proximal part of the catheter head (If) remaining on the body surface and having an RFID tag or transponder/data memory. This embodiment permits identification of the proximal part of the infusion set (1) even when the catheter tube is not connected. In the embodiment shown, a remote-coupling RFID system is used that permits a range of up to 1 m. The distal part of the catheter head (1e) remains connected (1b) with the catheter tube (1b) on the administration device. This embodiment permits an identification of the infusion set (1) even when the catheter tube is not connected. In addition, this embodiment affords a possibility of determining if the catheter tube and catheter head are no longer present in a configuration that conforms with the therapy, for example an expired catheter tube part (tube apparatus) and a new distal catheter head part (head apparatus). Likewise, secondary priming is possible with the priming volume of the distal catheter head part (head apparatus) if the head apparatus (5a) has been exchanged too early.

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that may or may not be utilized in any embodiment of the present invention.

For the purposes of describing and defining the present invention it is noted that the term “substantially” is utilized herein to represent the inherent degree of uncertainty that may be attributed to any quantitative comparison, value, measurement, or other representation. The term “substantially” is also utilized herein to represent the degree by which a quantitative representation may vary from a stated reference without resulting in a change in the basic function of the subject matter at issue.

While embodiments of the present invention have been illustrated and described in detail in the foregoing drawings and description, the same is to be considered as illustrative and not restrictive in character, it being understood that illustrative embodiments thereof have been shown and described and that all changes and modifications that come within the spirit of the present invention are desired to be protected.

1. A fluid delivery system for infusing a medication into a body of an animal, the system comprising:
an infusion device for dispensing the medication, wherein the infusion device comprises a data exchange device;
an infusion set connectable to the infusion device for administering the medication into the body of an animal, wherein the infusion set comprises a data memory for exchanging data with the infusion device via the data exchange device;
wherein when the data present on the data memory is transmitted to the infusion device via a data exchange device, the infusion device configured to identify deviations from an operating state, and when no deviations are detected, the operating of the infusion device or the infusion set can be established in one of an automated manner or manually.

2. The system as claimed in claim 1, wherein the deviation from an operating state include is one or more of: a leak in the fluid channel of the infusion set, an occlusion in the fluid channel of the infusion set, a connection between the infusion set and the infusion device has not been made correctly, a connection between head and tube of the catheter head has not been made correctly, or the infusion set does not sit correctly on the body

3. The system as claimed in claim 1, wherein the data memory comprises at least one overwritable and/or at least one non-overwritable data area.

4. The system as claimed in claim 3, wherein the data on the overwritable data areas can be influenced by an authorized administration device.

5. The system as claimed in claim 3, wherein the data on the overwritable data areas is configured such that the data may be altered by an output signal from at least one status detector located on the infusion set.

6. The system as claimed in claim 5, wherein the status detector located on the infusion set comprises one or more of: an occlusion sensor, a leakage sensor, a connection sensor, or a priming sensor.

7. The system as claimed in claim 5, wherein the output signal from a status detector is stored as a status flag in the overwritable data area.

8. The system as claimed in claim 3, wherein the non-overwritable data area comprises at least one type-specific identifier that defines a specific priming volume or the non-overwritable data enables the determination of a specific priming volume, such that an automated or semi-automated priming procedure can be instigated.

9. The system as claimed in claim 1, wherein the data exchange device comprises wireless transmission technology.

10. The system as claimed in claim 9, wherein the data exchange device comprises wireless RFID technology.

11. The system of claim 1, wherein the infusion set further comprises a catheter head insertable into the body and a connection end connectable to the infusion device and a tubing between the head and the connection end.

12. The system as claimed in claim 11, wherein an RFID memory/transponder unit is situated on the catheter head and/or on the connection part and/or on the tubing.

13. The system as claimed in claim 1, wherein the data memory arranged on the infusion set communicates with the infusion device via a direct electrical connection on the connector.

14. The system as claimed in claim 1, wherein the data memory arranged on the infusion set communicates with the administration device by an optical transmission method via the data exchange device.

15. The system as claimed in claim 1, wherein the data memory arranged on the infusion set communicates with the administration device by a unidirectional optical method.

16. The system as claimed in claim 1, wherein the data memory arranged on the infusion set communicates with the administration device via mechanical coding cams on the connector.

17. A method for infusing a medication into a body of an animal, the method comprising:
providing an infusion device for dispensing the medication, wherein the infusion device comprises a data exchange device;
providing an infusion set connectable to the infusion device for administering the medication into the body of an animal, wherein the infusion set comprises a data memory for exchanging data with the infusion device via the data exchange device;
transmitting the data on the data memory to the infusion device via the data exchange device;
identifying deviations of the configuration of the infusion set to a pre-defined therapy; and
establishing automatically or manually an operation state of the infusion set when no deviations are identified.

18. A method for exchanging data between an infusion set that infuses medication into a body of an animal and is connectable to an infusion device holding the medication, the method comprising:
providing a data exchange device on the infusion device;
providing a data memory on the infusion set for exchanging data with the infusion device via the data exchange device;
transmitting the data on the data memory to the infusion device via the data exchange device;
identifying deviations of the configuration of the infusion set;
indicating the configuration of the infusion set conforms to pre-defined therapy; and
establishing automatically or manually an operation state of the infusion set when no deviations are identified.

19. The method as claimed in claim 18, further comprising:
identifying memory data that defines a priming volume of an infusion set or determining a priming volume of an infusion set; and
initiating an automatic or semi-automatic priming procedure in the infusion device.

20. The method as claimed in claim 18, further comprising invalidating the infusion device via the data exchange device after expiry of the wearing or use period of an infusion set attached to an administration device, or writing the expiry of the infusion device on the data memory.

21. The method as claimed in claim 18, further comprising:
identifying via an attached connection detector an incorrect connection between infusion set and administration device;
prompting a user to rectify the incorrect connection; and
preventing use until the incorrect connection has been corrected.

22. The method as claimed in claim 18, further comprising:
detecting an occlusion in the infusion set via an attached occlusion detector;
identifying data memory information corresponding to the detected occlusion; and
prompting a user to rectify the occlusion.

23. The method as claimed in claim 18, further comprising:
detecting a leakage in the infusion set via an attached leakage detector;
identifying data memory information corresponding to the detected leak; and
prompting a user to rectify the causes of the leak.
